(19) United States
(12) Patent Application Publication
(10) Pub. No.: US 2009/0258405 A1
(43) Pub. Date: Oct. 15, 2009

(54) Preparation of optically active alcohols with whole-cell catalysts

(75) Inventors: Harald Groeger, Hanau (DE); Oliver May, Aachen (DE); Claudia Rollmann, Alzenau (DE); Francoise Chamouleau, L'Isle d'Espagnac (FR); Nicolas Orologas, Thessaloniki (GR); Karlheinz Drauz, Freigericht (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: DEGUSSA AG., Duesseldorf (DE)

(21) Appl. No.: 11/629,407

(22) PCT Filed: Jun. 9, 2005
(86) PCT No.: PCT/EP05/06215

§ 371 Date: Oct. 10, 2007

(30) Foreign Application Priority Data
Jun. 14, 2004 (DE) 10 2004 028 407.5

(51) Int. Cl. C12P 7/02

(52) U.S. Cl. 435/155

(57) ABSTRACT

The present invention relates to a process for the preparation of optically active alcohols from ketones with the aid of whole-cell catalysts comprising an alcohol dehydrogenase and also an enzyme capable of cofactor regeneration, a substrate concentration of at least 500 mM of ketone being provided for the conversion and the conversion being carried out without the addition of an “external” cofactor.

The present invention relates to a process for the preparation of optically active alcohols, starting from ketones, in the presence of a whole-cell catalyst comprising an alcohol dehydrogenase and also an enzyme capable of cofactor regeneration, which process is distinguished in that it is carried out at high substrate concentrations of >500 mM (without the addition of a cofactor).

The preparation of optically active alcohols is of interest, for example, for the pharmaceuticals industry and the foodstuffs industry. A preferred form of preparation is to obtain the optically active alcohols by reduction of ketones in the presence of alcohol dehydrogenases. This enzymatic reduction of ketones has already been described in detail in the literature. For example, reference may be made here to the overview articles by M.-R. Kula, U. Kragl, Dehydrogenases in the Synthesis of Chiral Compounds in Stereoselective Biocatalysis (ed.: R. N. Patel), Dekker, 2000, Chapter 28, p. 839-866 and J. D. Stewart, Dehydrogenases and Transaminases in Asymmetric Synthesis in Current Opinion in Chemical Biology 2001, 5, 120-129. Also known in this connection are methods of “regenerating” the cofactor consumed during the reaction. A particularly interesting method of regenerating the cofactors NAD⁺ or NADP⁺ consists in using a second dehydrogenase enzyme, in particular a formate dehydrogenase or a glucose dehydrogenase. The use of a whole-cell catalyst for the reduction has proved to be a preferred form of implementation because—in contrast to the use of isolated enzymes in purified form or in the form of their crude extract—there are no additional costs for cell opening and enzyme purification. Likewise preferred is the use of recombinant expression systems, because high rates of expression can be achieved therewith. In comparison with non-recombinant cells, for example baker's yeast, such recombinant whole-cell catalysts can correspondingly be used in smaller amounts. A further advantage, in addition to higher reaction rates, is the avoidance of undesirable secondary reactions by further dehydrogenase enzymes contained in wild-type cells. The advantages of the whole-cell method using microorganisms which have been genetically modified by means of recombinant DNA technology are described in detail, inter alia, in M. Kataoka, K. Kita, M. Wada, Y. Yasohara, J. Hasegawa, S. Shimizu, Appl. Microbiol. Biotechnol. 2003, 62, 437-445. In particular, E. coli cells that express an alcohol dehydrogenase and a glucose dehydrogenase for cofactor regeneration have proved to be especially suitable.

Reductions with high, commercially attractive substrate concentrations have proved to be a particular challenge. Using a whole-cell catalyst in which an ADH and a glucose dehydrogenase were present it has been possible in a two-phase reaction system to produce optically active alcohols even with high substrate concentrations of >500 mM. This has been demonstrated in particular for the preparation of (S)-4-chloro-3-hydroxybutanoic acid ethyl ester. However, it was necessary to add a cofactor, in this case NADP⁺, for the preparation of these alcohols. As described, for example, in N. Kizaki, Y. Yasohara, J. Hasegawa, M. Wada, M. Kataoka, S. Shimizu, Appl. Microbiol. Biotechnol. 2001, 55, 590-595, the added amount of NADP⁺ was in the region of about 0.001 mol. equivalent, based on substrate used. Because of the high price of NADP⁺, the added cofactor makes a significant contribution to the overall cost of the process even with these relatively small amounts of cofactor. Accordingly, a process that dispenses with the “external addition” of cofactor would be advantageous.

Whole-cell transformations (conversion of a substrate by means of whole cells), based on the activity of an alcohol dehydrogenase and a formate dehydrogenase as cofactor-regenerating enzyme, without the addition of cofactor, have recently been described in A. Matsuyama, H. Yamamoto, Y. Kobayashi, Organic Process Research & Development 2002, 6, 558-561. However, the substrate concentrations used were below 250 mM (e.g. 196 mM and 217 mM), and long reaction times of from 17 to 48 hours were required. Corresponding biotransformations with substantially higher substrate concentrations and short reaction times (less than 10 hours) are not known.

The significance of the addition of cofactor for the whole-cell method is also described in N. Itoh, M. Matsuda, M. Mabuchi, T. Dairi, J. Wang, Eur. J. Biochem. 2002, 269, 2394-2402. It was found here that a satisfactory conversion was achieved with the addition of only 0.5 mM, whereas scarcely any product formation took place without the addition of NAD⁺. In this connection Itoh et al. found that “the endogenous NAD+/NADH in the E. coli cells was insufficient for a smooth reaction”, accompanied by the necessity for the “external” addition of cofactor.

The object of the present invention was, therefore, to develop a rapid, simple, inexpensive and effective process for the preparation of optically active alcohols from ketones.

The object has been achieved according to the invention by a process for the preparation of optically active alcohols by reduction of ketones in the presence of a whole-cell catalyst comprising an alcohol dehydrogenase and also an enzyme capable of cofactor regeneration, characterised in that the conversion of a substrate concentration of at least 500 mM per starting volume of aqueous solvent used is carried out without the addition of an “external” cofactor. According to the invention this is to be understood as meaning that at least 500 mM of the substrate are converted by means of the described process per starting volume of aqueous solvent (including buffer system) used. It can be left open whether the at least 500 mM of substrate are actually achieved as concentration in the reaction mixture, or whether a substrate concentration of at least 500 mM is converted in total, based on the starting volume of aqueous solvent. The process is furthermore particularly suitable for the reduction of ketones using substrate concentrations of >500 mM, preferably >1000 mM and very preferably >1500 mM of ketone.

However, very particular preference is given to the variant in which a substrate concentration of at least 500 mM of ketone is actually provided for the conversion. The concentrations referred to here relate to concentrations of the substrate (ketone), based on the starting volume of aqueous solvent, that are actually achieved in the batch, it being immaterial when this starting concentration is achieved in the course of the period of incubation of a whole-cell catalyst that is used. The ketone can be used in these concentrations in the form of a batch directly at the start of a whole-cell batch, or a whole-cell catalyst can first be employed to a particular optical density, before the ketone is added. Likewise, the ketone can first be used in lower concentrations and added in the course of the incubation period of the cell batch to concentrations as indicated. According to the invention, however, a concentration of substrate (ketone) of at least 500 mM is achieved in the cell batch at least once during the conversion of the substrate to the desired alcohol.

Using this process, high to very high conversions to the corresponding optically active alcohol of at least 80%, especially >90% and very preferably >95% are surprisingly achieved at high substrate concentrations of at least 500 mM of ketone, especially >750 mM and very preferably >1000 mM even without the addition of an “external” cofactor. This was not to be expected on the basis of the conversions known hitherto and the known problems of the diffusion of the cofactor as a result of the permeabilisation of the cell membrane under the reaction conditions. This is additionally especially surprising because, at the high substrate concentrations of at least 500 mM of hydrophobic ketone component and in view of the low cell concentrations of the biocatalyst of <75 g/l, preferably <50 g/l, permeabilisation of the cell membrane should occur to a particularly great extent, accompanied by a loss of intracellular cofactor by “washing out” of the cofactor into the reaction medium.

The addition of the ketone can be carried out in any desired manner. Preferably, the total amount of ketone is added at the beginning (“batch” method), or alternatively it is added in metered amounts. It is also possible to employ continuous addition (“continuous feed-in process”).

According to the invention, the process described here for the preparation of optically active alcohols is used. The conversion of ketones to optically active alcohols with the aid of alcohol dehydrogenases is known in principle to the person skilled in the art (see the literature references mentioned above). In order to obtain optically active alcohols it is particularly preferred to use ketones whose substituents are different from one another. Examples of optically active alcohols which can be prepared from the corresponding ketones are likewise known to the person skilled in the art. They can be subsumed under the following general formula

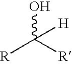

in which R and R′ are different from one another and are (C₁-C₈) alkyl, (C₁-C₈)-alkoxy, HO-(C₁-C₈)-alkyl, (C₂-C₈)-alkoxyalkyl, (C₆-C₁₈)-aryl, (C₇-C₁₉)-aralkyl, (C₃-C₁₈)-heteroaryl, (C₄-C₁₉)-heteroaralkyl, (C₁-C₈)-alkyl-(C₆-C₁₈)-aryl, (C₁-C₈)-alkyl-(C₃-C₁₈)-heteroaryl, (C₃-C₈)-cycloalkyl, (C₁-C₈)-alkyl-(C₃-C₈)-cycloalkyl, (C₃-C₈)-cycloalkyl-(C₁-C₈)-alkyl.

The concentration of biocatalyst is not more than 75 g/l, in a preferred embodiment up to 50 g/l, preferably up to 25 g/l and particularly preferably up to 15 g/l, g being based on g of bio wet mass (BWM). The biocatalyst is to be understood as being especially a whole-cell catalyst.

In a preferred embodiment, the conversion of the ketone to the desired optically active alcohol is carried out without the addition of an organic solvent. This is intended to mean that no organic solvent is added to the batch containing the biocatalyst.

It is additionally preferred to carry out the conversion in a cell suspension of the suitable whole-cell catalyst, it being possible for the ketone used likewise to be present in the form of a suspension in the cell suspension or in the form of an emulsion or solution in the cell suspension.

For the present invention, one of the genes preferably to be selected is a gene for an alcohol dehydrogenase. The person skilled in the art is likewise free to choose the genes that code for such an alcohol dehydrogenase. Examples of alcohol dehydrogenases that have proved to be preferable are alcohol dehydrogenases from a Lactobacillus strain, especially from Lactobacillus kefir and Lactobacillus brevis, or alcohol dehydrogenases from a Rhodococcus strain, especially from Rhodococcus erythropolis and Rhodococcus ruber, or alcohol dehydrogenases from an Arthrobacter strain, especially from Arthrobacter paraffineus.

A further gene that is particularly preferred for the present invention is a gene that codes for a dehydrogenase. Here too, the person skilled in the art is free to choose the genes that code for a dehydrogenase capable of cofactor regeneration. Preferred dehydrogenases for cofactor regeneration have proved to be glucose dehydrogenases, preferably a glucose dehydrogenase from Bacillus, Thermoplasma and Pseudomonas strains, or formate dehydrogenases, preferably a formate dehydrogenase from Candida and Pseudomonas strains, or malate dehydrogenases (“malic enzyme”), preferably a malic enzyme from Sulfolobus, Clostridium, Bacillus and Pseudomonas strains as well as from E. coli, especially E. coli K12.

According to the present invention, a “whole-cell catalyst” is to be understood as being an intact cell in which at least one gene is expressed that is able to catalyse the conversion according to the invention of a substrate to a product. According to the invention, the intact cell is capable of expressing an alcohol dehydrogenase and a dehydrogenase capable of cofactor regeneration. The whole-cell catalyst is preferably a genetically modified microorganism adapted to the requirements of the desired conversion. Preference is given as particularly suitable whole-cell catalysts to the two whole-cell catalysts described in the experimental part.

For the whole-cell catalyst, containing an alcohol dehydrogenase and an enzyme capable of cofactor regeneration, all known cells are suitable. There may be mentioned as microorganisms in this connection organisms such as, for example, yeasts such as Hansenula polymorpha, Pichia sp., Saccharomyces cerevisiae, prokaryotes, such as E. coli, Bacillus subtilis, or eukaryotes, such as mammalian cells, insect cells or plant cells. The cloning methods are well known to the person skilled in the art (Sambrook, J.; Fritsch, E. F. and Maniatis, T. (1989), Molecular cloning: a laboratory manual, 2^{nd} ed., Cold Spring Harbor Laboratory Press, New York). E. coli strains are preferably to be used for this purpose. Very particular preference is given to: E. coli XL1 Blue, NM 522, JM101, JM109, JM105, RR1, DH5α, TOP 10- , HB101, BL21 codon plus, BL21 (DE3) codon plus, BL21, BL21 (DE3), MM294. Plasmids with which the gene construct containing the nucleic acid according to the invention is preferably cloned into the host organism are likewise known to the person skilled in the art (see also PCT/EP03/07148; see below). Suitable plasmids or vectors are in principle any forms available to the person skilled in the art for this purpose. Such plasmids and vectors can be found, for example, in Studier et al. (Studier, W. F.; Rosenberg A. H.; Dunn J. J.; Dubendroff J. W.; (1990), Use of the T7 RNA polymerase to direct expression of cloned genes, Methods Enzymol. 185, 61-89) or the brochures of Novagen, Promega, New England Biolabs, Clontech or Gibco BRL. Further preferred plasmids and vectors can be found in: Glover, D. M. (1985), DNA cloning: a practical approach, Vol. I-III, IRL Press Ltd., Oxford; Rodriguez, R. L. and Denhardt, D. T (eds) (1988), Vectors: a survey of molecular cloning vectors and their uses, 179-204, Butterworth, Stoneham; Goeddel, D. V. (1990), Systems for heterologous gene expression, Methods Enzymol. 185, 3-7; Sambrook, J.; Fritsch, E. F. and Maniatis, T. (1989), Molecular cloning: a laboratory manual, 2^{nd} ed., Cold Spring Harbor Laboratory Press, New York.

Plasmids with which the gene constructs containing the nucleic acid sequences under consideration can very preferably be cloned into the host organism are or are based on: pUC18/19 (Roche Biochemicals), pKK-177-3H (Roche Biochemicals), pBTac2 (Roche Biochemicals), pKK223-3 (Amersham Pharmacia Biotech), pKK-233-3 (Stratagene) or pET (Novagen).

In a further embodiment of the process according to the invention, the whole-cell catalyst is preferably pretreated before use in such a manner that the permeability of the cell membrane to the substrates and products is increased compared with the intact system. Particular preference is given to a process in which the whole-cell catalyst is pretreated, for example, by freezing and/or treatment with toluene.

According to the invention, the process can be carried out without the addition of an “external” cofactor. This means that it is not necessary to add additional cofactor to the whole-cell batch, because the cells themselves already contain and are able to use a cofactor suitable for the conversion reaction. Cofactors suitable for the conversion are to be understood as being those to which electrons can be transferred, such as, for example, the NAD(P)+ H⁺ and the FADH₂ system.

The process according to the invention can be carried out at any reaction temperatures suitable for the host organism used. A particularly suitable reaction temperature is considered to be a reaction temperature that is from 10 to 90° C., preferably from 15 to 50° C. and particularly preferably from 20 to 350C.

The person skilled in the art is also free to choose the pH value of the reaction, it being possible to carry out the reaction both at a fixed pH value and with variation of the pH value within a pH range. The pH value is chosen taking into account in particular the needs of the host organism that is used. Preferably, the reaction is carried out at a pH value from pH 5 to 9, preferably from pH 6 to 8 and particularly preferably from pH 6.5 to 7.5.

The conversion of the substrate used to the desired product is carried out in cell culture using a suitable whole-cell catalyst. A suitable nutrient medium is employed according to the host organism that is used. The media suitable for the host cells are generally known and commercially available. It is additionally possible to add to the cell cultures conventional additives such as, for example, antibiotics, growth-promoting agents such as, for example, serums (foetal calf serum, etc.) and similar known additives.

(C₁-C₈)-Alkyl radicals are to be regarded as being methyl, ethyl, n-propyl, isopropyl, n-butyl, isobutyl, sec-butyl, tert-butyl, pentyl, hexyl, heptyl or octyl, including all their bond isomers.

The radical (C₁-C₈)-alkoxy corresponds to the radical (C₁-C₈)-alkyl, with the proviso that it is bonded to the molecule via an oxygen atom.

(C₂-C₈)-Alkoxyalkyl means radicals in which the alkyl chain is interrupted by at least one oxygen function, wherein two oxygen atoms may not be bonded to one another. The number of carbon atoms indicates the total number of carbon atoms contained in the radical.

A (C₃-C₅)-alkylene bridge is a carbon chain having from three to five carbon atoms, the chain being bonded to the molecule under consideration via two different carbon atoms.

The radicals just described may be mono- or poly-substituted by halogens and/or by radicals containing N, O, P, S, Si atoms. These are especially alkyl radicals of the above-mentioned type, which contain one or more of these hetero atoms in their chain or which are bonded to the molecule via one of these hetero atoms.

(C₃-C₈)-Cycloalkyl is understood as being cyclopropyl, cyclobutyl, cyclopentyl, cyclohexyl or cycloheptyl radicals, etc. These radicals may be substituted by one or more halogens and/or radicals containing N, O, P, S, Si atoms and/or may contain N, O, P, S atoms in the ring, such as, for example, 1-, 2-, 3-, 4-piperidyl, 1-, 2-, 3-pyrrolidinyl, 2-, 3-tetrahydrofuryl, 2-, 3-, 4-morpholinyl.

A (C₃-C₈)-cycloalkyl-(C₁-C₈)-alkyl radical denotes a cycloalkyl radical as described above which is bonded to the molecule via an alkyl radical as indicated above.

Within the scope of the invention, (C₁-C₈)-acyloxy means an alkyl radical as defined above which has not more than 8 carbon atoms and is bonded to the molecule via a COO function.

Within the scope of the invention, (C₁-C₈)-acyl means an alkyl radical as defined above which has not more than 8 carbon atoms and is bonded to the molecule via a CO function.

A (C₆-C₁₈)-aryl radical is understood as being an aromatic radical having from 6 to 18 carbon atoms. Such radicals include in particular compounds such as phenyl, naphthyl, anthryl, phenanthryl, biphenyl radicals or systems of the above-described type fused to the molecule in question, such as, for example, indenyl systems, which may optionally be substituted by (C₁-C₈)-alkyl, (C₁-C₈)-alkoxy, NR¹R², (C₁-C₈) -acyl, (C₁-C₈) -acyloxy.

A (C₇-C₁₉)-aralkyl radical is a (C₆-C₁₈)-aryl radical bonded to the molecule via a (C₁-C₈)-alkyl radical.

Within the scope of the invention, a (C₃-C₁₈)-heteroaryl radical denotes a five-, six- or seven-membered aromatic ring system of from 3 to 18 carbon atoms which contains hetero atoms such as, for example, nitrogen, oxygen or sulfur in the ring. Such heteroaromatic compounds are regarded as being in particular radicals such as 1-, 2-, 3-furyl, 1-, 2-, 3-pyrrolyl, 1-, 2-, 3-thienyl, 2-, 3-, 4-pyridyl, 2-, 3-, 4-, 5-, 6-, 7-indolyl, 3-, 4-, 5-pyrazolyl, 2-, 4-, 5-imidazolyl, acridinyl, quinolinyl, phenanthridinyl, 2-, 4-, 5-, 6-pyrimidinyl.

A (C₄-C₁₉)-heteroaralkyl is understood as being a heteroaromatic system corresponding to the (C7-C₁₉)-aralkyl radical.

Suitable halogens (Hal) are fluorine, chlorine, bromine and iodine.

The term aqueous solvent is understood as meaning water or a solvent mixture consisting mainly of water with water-soluble organic solvents such as, for example, alcohols, especially methanol or ethanol, or ethers, such as THF or dioxane.

### FIGURES

FIG. 1 shows the plasmid map of plasmid pNO5c

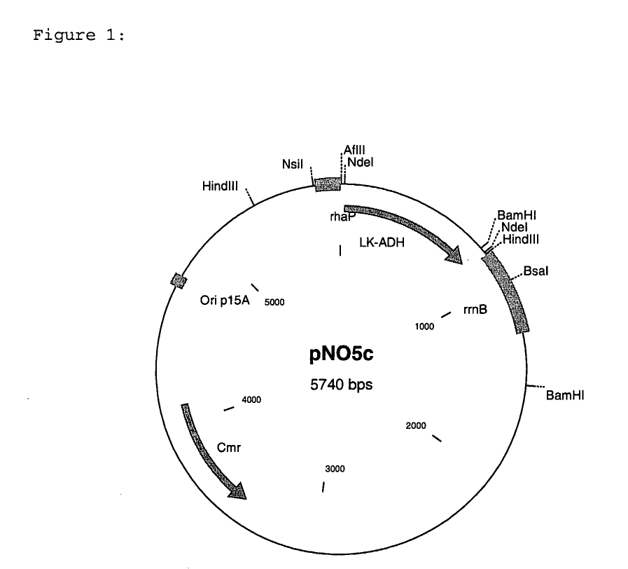

FIG. 2 shows the plasmid map of plasmid pNO8c

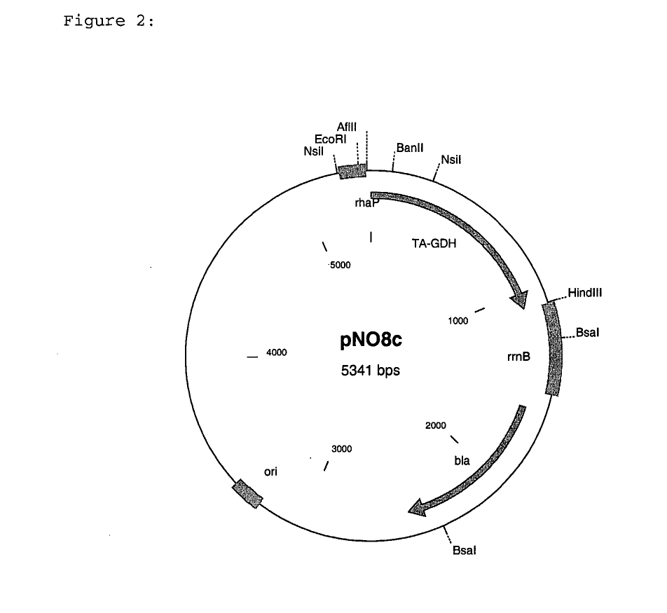

FIG. 3 shows the plasmid map of plasmid pNO14c

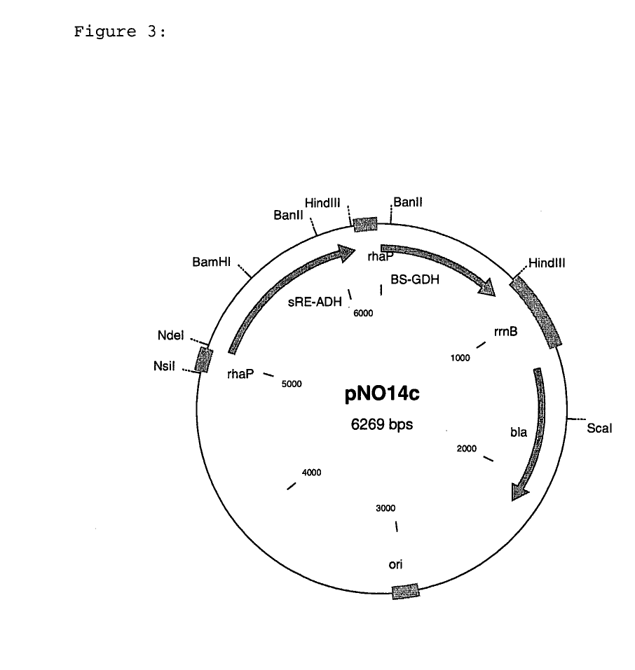

### EXAMPLES

### Preparation of a Whole-Cell Catalyst Comprising an (R)-Alcohol Dehydrogenase from Lactobacillus kefir and a Glucose Dehydrogenase from Thermoplasma acidophilum

Preparation of the Strain

Chemically competent cells of E. coli DSM14459 (described in WO03/042412) were transformed with the plasmid pNO5c (Sambrook et al. 1989, Molecular cloning: A Laboratory Manual, 2nd Edition, Cold Spring Harbor Laboratory Press). This plasmid codes for the alcohol dehydrogenase from Lactobacillus kefir (Lactobacillus kefir alcohol dehydrogenase: a useful catalyst for synthesis. Bradshaw et al. JOC 1992, 57 1532-6, Reduction of acetophenone to R(+)-phenylethanol by a new alcohol dehydrogenase from Lactobacillus kefir. Hummel W. Ap Microbiol Biotech 1990, 34, 15-19). The recombinant strain E. coli DSM14459 (pNO5c) so prepared was made chemically competent and transformed with the plasmid pNO8c, which codes for the gene of a codon-optimised glucose dehydrogenase from Thermoplasma acidophilum (Bright, J. R. et al., 1993 Eur. J. Biochem. 211:549-554). Both genes are under the control of a rhamnose promoter (Stumpp, Tina; Wilms, Burkhard; Altenbuchner, Josef. A new, L-rhamnose-inducible expression system for Escherichia coli. BIOspektrum (2000), 6(1), 33-36). The sequences and plasmid maps of pNO5c and pNO8c are shown hereinbelow.

Preparation of Active Cells

An individual colony of E. coli DSM14459 (pNO5c,pNO8c) was incubated in 2 ml of LB medium with added antibiotic (50 μg/l ampicillin and 20 μg/ml chloramphenicol) for 18 hours at 37° C., with shaking (250 rpm). This culture was diluted 1:100 in fresh LB medium with rhamnose (2 g/l) as inducer, added antibiotic (50 μg/l ampicillin and 20 μg/ml chloramphenicol) and 1 mM ZnCl₂ and was incubated for 18 hours at 30° C., with shaking (250 rpm). The cells were then harvested by centrifugation (10,000 g, 10 min., 4° C.), the supernatant was discarded, and the cell pellet was used in biotransformation tests either directly or after storage at −20° C.

### Preparation of a Whole-Cell Catalyst Comprising an (S)-Alcohol Dehydrogenase from Rhodococcus erythropolis and a Glucose Dehydrogenase from Bacillus subtilis

Preparation of the Strain

Chemically competent cells of E. coli DSM14459 (described in WO03/042412) were transformed with the plasmid pNO14c (Sambrook et al. 1989, Molecular cloning: A Laboratory Manual, 2nd Edition, Cold Spring Harbor Laboratory Press). This plasmid codes for an alcohol dehydrogenase from Rhodococcus erythropolis (Cloning, sequence analysis and heterologous expression of the gene encoding a (S)-specific alcohol dehydrogenase from Rhodococcus erythropolis DSM 43297. Abokitse, K.; Hummel, W. Applied Microbiology and Biotechnology 2003, 62 380-386) and a glucose dehydrogenase from Bacillus subtilis (Glucose dehydrogenase from Bacillus subtilis expressed in Escherichia coli. I: Purification, characterization and comparison with glucose dehydrogenase from Bacillus megaterium. Hilt W; Pfleiderer G; Fortnagel P Biochimica et biophysica acta (1991 Jan. 29), 1076(2), 298-304). The alcohol dehydrogenase is under the control of a rhamnose promoter (Stumpp, Tina; Wilms, Burkhard; Altenbuchner, Josef. A new, L-rhamnose-inducible expression system for Escherichia coli. BIOspektrum (2000), 6(1), 33-36). The sequence and plasmid map of pNO14c is shown hereinbelow.

Preparation of Active Cells

An individual colony of E. coli DSM14459 (pNO14c) was incubated in 2 ml of LB medium with added antibiotic (50 μg/l ampicillin and 20 μg/ml chloramphenicol) for 18 hours at 37° C., with shaking (250 rpm). This culture was diluted 1:100 in fresh LB medium with rhamnose (2 g/l) as inducer, added antibiotic (50 μg/l ampicillin and 20 μg/ml chloramphenicol) and 1 mM ZnCl₂ and was incubated for 18 hours at 30° C., with shaking (250 rpm). The cells were harvested by centrifugation (10,000 g, 10 min., 4° C.), the supernatant was discarded, and the cell pellet was used in biotransformation tests either directly or after storage at −20° C.

### Synthesis Example 1

### Reduction of p-chloroacetophenone in a 0.5 M Solution Using a Whole-Cell Catalyst Comprising an (R)-Selective Alcohol Dehydrogenase

In a Titrino reaction vessel there are added to 50 ml of a phosphate buffer (adjusted to pH 7.0) at room temperature the above-described whole-cell catalyst E. coli DSM14459 (pNO5c,pNO8c) with an (R)-selective alcohol dehydrogenase (E. coli, (R)-alcohol dehydrogenase from L. kefir, glucose dehydrogenase from T. acidophilum) in a cell concentration of 25 g BWM/1, 1.5 equivalents of glucose (equivalents are based on the amount of p-chloroacetophenone used) and 25 mmol. of p-chloroacetophenone (corresponding to a substrate concentration, based on phosphate buffer used, of 0.5 M). The reaction mixture is stirred for 7 hours at room temperature, the pH being kept constant by the addition of sodium hydroxide solution (1M NaOH). Samples are taken at regular intervals, and the conversion of the p-chloroacetophenone is determined by means of HPLC. After a reaction time of 7 hours, the conversion is >99%.

### Synthesis Example 2

### Reduction of p-chloroacetophenone in a 0.5 M Solution Using a Whole-Cell Catalyst Comprising an (S)-Selective Alcohol Dehydrogenase

In a Titrino reaction vessel there are added to 50 ml of a phosphate buffer (adjusted to pH 7.0) at room temperature the above-described whole-cell catalyst E. coli DSM14459 (pNO14c) with an (S)-selective alcohol dehydrogenase (E. coli, (S)-alcohol dehydrogenase from R. erythropolis, glucose dehydrogenase from B. subtilis) in a cell concentration of 50 g BWM/1, 6 equivalents of glucose (equivalents are based on the amount of p-chloroacetophenone used) and 25 mmol. of p-chloroacetophenone (corresponding to a substrate concentration, based on phosphate buffer used, of 0.5 M). The reaction mixture is stirred for 7.5 hours at room temperature, the pH being kept constant by the addition of sodium hydroxide solution (1M NaOH). Samples are taken at regular intervals, and the conversion of the p-chloroacetophenone is determined by means of HPLC. After a reaction time of 7.5 hours, the conversion is 92%.

### Synthesis Example 3

### Reduction of Acetophenone in a 1.5 M Solution Using a Whole-Cell Catalyst Comprising an (R)-Selective Alcohol Dehydrogenase

In a Titrino reaction vessel there are added to 40 ml of a phosphate buffer (adjusted to pH 7.0) at room temperature the above-described whole-cell catalyst E. coli DSM14459 (pNO5c, pNO8c) with an (R)-selective alcohol dehydrogenase (E. coli, (R)-alcohol dehydrogenase from L. kefir, glucose dehydrogenase from T. acidophilum) in a cell concentration giving an optical density of OD=21.15, 1.05 equivalents of glucose (equivalents are based on the amount of acetophenone used) and 60 mmol. of acetophenone (corresponding to a substrate concentration, based on phosphate buffer used, of 1.5 M). The reaction mixture is stirred for 23 hours at room temperature, the pH being kept constant by the addition of sodium hydroxide solution (2M NaOH). Samples are taken at regular intervals, and the conversion of the acetophenone is determined by means of HPLC. After reaction times of 16.5 and 23 hours, the conversion is 93% and 97%, respectively.

1. Process for the preparation of optically active alcohols by reduction of ketones in the presence of a whole-cell catalyst comprising an alcohol dehydrogenase and also an enzyme capable of cofactor regeneration, wherein the conversion of a substrate concentration of at least 500 mM per starting volume of aqueous solvent used is carried out without the addition of an “external” cofactor.

2. Process according to claim 1, wherein a substrate concentration of at least 500 mM of ketone is provided for the conversion.

3. Process according to claim 1, wherein the concentration of biocatalyst used does not exceed 75 g/l.

4. Process according to claim 1, wherein the process is carried out in the absence of an organic solvent.

5. Process according to claim 1, wherein there is used a whole-cell catalyst comprising at least one alcohol dehydrogenase selected from the group consisting of an alcohol dehydrogenase from a Lactobacillus strain, especially from Lactobacillus kefir and Lactobacillus brevis, and/or an alcohol dehydrogenase from a Rhodococcus strain, especially from Rhodococcus erythropolis and Rhodococcus ruber, and/or an alcohol dehydrogenase from an Arthrobacter strain, especially from Arthrobacter paraffineus.

6. Process according to claim 1, wherein the whole-cell catalyst comprises as an enzyme capable of cofactor regeneration a glucose dehydrogenase, preferably from Bacillus, Thermoplasma and Pseudomonas strains.

7. Process according to claim 1, wherein the whole-cell catalyst comprises as an enzyme capable of cofactor regeneration a formate dehydrogenase, preferably from Candida and Pseudomonas strains.

8. Process according to claim 1, wherein the whole-cell catalyst comprises as an enzyme capable of cofactor regeneration a malate dehydrogenase.

9. Process according to claim 1, wherein the reaction temperature is from 10 to 90° C., preferably from 15 to 50° C. and very preferably from 20 to 35° C.

10. Process according to claim 1, wherein the pH value is from pH 5 to 9, preferably from pH 6 to 8 and particularly preferably from 6.5 to 7.5.

11. Process according to claim 1, wherein the total amount of substrate is added at the beginning.

12. Process according to claim 1, wherein the substrate is metered in during the reaction.


SEQUENCE LISTING (3 sequences)

SEQ ID NO: 1 - DNA, 5740 bp, Artificial Sequence
  pNO5c with Lactobacillus kefir -ADH
aattcttaagaaggagatatacatatgactgatcgtttaaaaggcaaagtagcaattgtaactggcggtaccttgggaattggcttggcaatcgctgataagtttgttgaagaaggcgcaaaggttgttattaccggccgtcacgctgatgtaggtgaaaaagctgccaaatcaatcggcggcacagacgttatccgttttgtccaacacgatgcttctgatgaagccggctggactaagttgtttgatacgactgaagaagcatttggcccagttaccacggttgtcaacaatgccggaattgcggtcagcaagagtgttgaagataccacaactgaagaatggcgcaagctgctctcagttaacttggatggtgtcttcttcggtacccgtcttggaatccaacgtatgaagaataaaggactcggagcatcaatcatcaatatgtcatctatcgaaggttttgttggtgatccaactctgggtgcatacaacgcttcaaaaggtgctgtcagaattatgtctaaatcagctgccttggattgcgctttgaaggactacgatgttcgggttaacactgttcatccaggttatatcaagacaccattggttgacgatcttgaaggggcagaagaaatgatgtcacagcggaccaagacaccaatgggtcatatcggtgaacctaacgatatcgcttggatctgtgtttacctggcatctgacgaatctaaatttgccactggtgcagaattcgttgtcgatggtggatacactgctcaataaggatccgaattcgagctcaggaggtatcatatgcagccaagcttctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggattagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcatcgatcaccacaattcagcaaattgtgaacatcatcacgttcatctttccctggttgccaatggcccattttcctgtcagtaacgagaaggtcgcgaattcaggcgctttttagactggtcgtaatgaac

SEQ ID NO: 2 - DNA, 5341 bp, Artificial Sequence
  pNO8c with Thermoplasma acidophilum - GDH
tatgaccgagcagaaagcgattgtgaccgatgcgccgaaaggtggtgtgaaatacaccaccattgatatgccggaaccggaacattatgatgcgaaactgagcccggtgtatatcggtatttgcggcaccgatcgtggtgaagtggcgggtgcgctgagctttacctataacccggaaggcgaaaactttctggtgctgggccatgaagcgctgctgcgtgtggatgatgcgcgtgataacggctatatcaaaaagggcgatctggtggtgccgctggtgcgtcgtccgggtaaatgcattaactgccgcattggccgtcaggataactgtagcattggcgatccggataaacatgaagcgggcattaccggcctgcatggctttatgcgcgatgtgatctatgatgatattgaatatctggtgaaagtggaagatccggaactgggtcgtattgcggtgctgaccgaaccgctgaaaaacgtgatgaaagcgtttgaagtgtttgatgtggtgagcaaacgcagcattttctttggcgatgatagcaccctgattggcaaacgcatggtgattatcggcagcggtagcgaagcgtttctgtatagctttgcgggcgtggatcgtggttttgatgtgaccatggtgaaccgccatgatgaaaccgaaaacaaactgaaaatcatggatgaatttggcgtgaaattcgcgaactatctgaaagatatgccggagaaaatcgatctgctggttgataccagcggtgatccgaccaccaccttcaaattcctgcgcaaagtgaacaacaacggcgtggtgattctgtttggcaccaacggtaaagcgccgggttatccggtggatggcgaagatattgattacattgtggaacgcaacattaccattgcgggtagcgtggatgcggcgaaaatccattatgtgcaggcgctgcaaagcctgagcaactggaatcgtcgtcatccggatgcgatgaaaagcattatcacctatgaagcgaaaccgagcgaaaccaacattttctttcagaaaccgcatggcgaaatcaaaaccgtgatcaaatggcagtaagcttctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtataccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcatcgatcaccacaattcagcaaattgtgaacatcatcacgttcatctttccctggttgccaatggcccattttcctgtcagtaacgagaaggtcgcgaattcaggcgctttttagactggtcgtaatgaacaattcttaagaaggagatataca

SEQ ID NO: 3 - DNA, 6269 bp, Artificial Sequence
  pNO14c with R. erythropolis - sADH and B. subtilis - GDH
tatgtatccggatttaaaaggaaaagtcgtcgctattacaggagctgcttcagggctcggaaaggcgatggccattcgcttcggcaaggagcaggcaaaagtggttatcaactattatagtaataaacaagatccgaacgaggtaaaagaagaggtcatcaaggcgggcggtgaagctgttgtcgtccaaggagatgtcacgaaagaggaagatgtaaaaaatatcgtgcaaacggcaattaaggagttcggcacactcgatattatgattaataatgccggtcttgaaaatcctgtgccatctcacgaaatgccgctcaaggattgggataaagtcatcggcacgaacttaacgggtgcctttttaggaagccgtgaagcgattaaatatttcgtagaaaacgatatcaagggaaatgtcattaacatgtccagtgtgcacgaagtgattccttggccgttatttgtccactatgcggcaagtaaaggcgggataaagctgatgacagaaacattagcgttggaatacgcgccgaagggcattcgcgtcaataatattgggccaggtgcgatcaacacgccaatcaatgctgaaaaattcgctgaccctaaacagaaagctgatgtagaaagcatgattccaatgggatatatcggcgaaccggaggagatcgccgcagtagcagcctggcttgcttcgaaggaagccagctacgtcacaggcatcacgttattcgcggacggcggtatgacacaatatccttcattccaggcaggccgcggttaatagtagaagcttctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtataggcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcatcgatcaccacaattcagcaaattgtgaacatcatcacgttcatctttccctggttgccaatggcccattttcctgtcagtaacgagaaggtcgcgaattcaggcgctttttagactggtcgtaatgaacaattcttaagaaggagatatacatatgaaagcgatccagtacacccgtattggtgcggaaccggaactgaccgaaatcccgaaaccggaaccgggtccgggtgaagttctgctggaagttaccgcggcgggtgtttgtcatagcgatgatttcatcatgagcctgccggaagaacagtatacctatggcctgccgctgaccctgggtcatgaaggtgcgggtaaagttgcggcggttggcgaaggtgttgaaggcctggatatcggcaccaacgtggtggtttatggtccgtggggttgcggtaattgctggcattgtagccagggcctggaaaactattgtagccgtgcgcaggaactgggcatcaatccgccgggtctgggtgcgccgggtgcgctggcggaattcatgattgtggatagcccgcgtcatctggttccgattggcgatctggatccggtgaaaaccgttccgctgaccgatgcgggtctgaccccgtatcatgcgatcaaacgcagcctgccgaaactgcgtggtggtagctatgcggtggttattggcaccggtggtctgggtcatgttgcgattcagctgctgcgtcatctgagcgcggcgaccgttattgcgctggatgtgagcgcggataaactggaactggcgaccaaagttggtgcgcatgaagtggtgctgagcgataaagatgcggcggaaaacgtgcgcaaaatcaccggtagccagggtgcggcgctggttctggattttgtgggctaccagccgaccattgataccgcgatggcggttgcgggtgttggtagcgatgtggcgattgtgggcattggtgatggtcaggcgcatgcgaaagtgggtttcttccagagcccgtatgaagcgagcgttaccgttccgtattggggtgcgcgcaacgaactgattgaactgatcgatctggcgcatgcgggcatctttgatatcgcggtggaaaccttcagcctggataatggcgcggaagcgtatcgtcgtctggcggcgggcaccctgagcggtcgtgcggttgttgttccgggtctgtaagcttctgcatcgatcaccacaattcagcaaattgtgaacatcatcacgttcatctttccctggttgccaatggcccattttcctgtcagtaacgagaaggtcgcgaattcaggcgctttttagactggtcgtaatgaacaattcttaagaaggagatataca